(19) United States
(12) Patent Application Publication
(10) Pub. No.: US 2013/0034604 A1
(43) Pub. Date: Feb. 7, 2013

(54) EXTENDED RELEASE FORMULATIONS OF DESVENLAFAXINE BASE

(71) Applicants: Veerababu Ramabrahmmam Taduri, Vadodara (IN); Girish Shantilal Achliya, Vadodara (IN); Divyakumar Hemant Bora, Vadodara (IN)

(72) Inventors: Veerababu Ramabrahmmam Taduri, Vadodara (IN); Girish Shantilal Achliya, Vadodara (IN); Divyakumar Hemant Bora, Vadodara (IN)

(73) Assignee: ALEMBIC PHARMACEUTICALS LIMITED, Vadodara (IN)

(21) Appl. No.: 13/642,429

(22) PCT Filed: May 9, 2011
(86) PCT No.: PCT/IB2011/000979

§ 371 Date: Oct. 19, 2012

(30) Foreign Application Priority Data
May 14, 2010 (IN) 1526/MUM/2010
Apr. 15, 2011 (IN) 1233/MUM/2011

(51) Int. Cl. A61K 9/22; A61P 25/24; A61K 9/16; A61K 31/137

(52) U.S. Cl. 424/468; 514/649; 427/2.21

(57) ABSTRACT

An extended release pharmaceutical composition comprising micronized desvenlafaxine base, at least one pH modifier and at least one release controlling agent and its process for preparation. An extended release monolithic tablet is also provided. Further extended release tablets comprising micronized desvenlafaxine base; at least one pH modifier; at least one release controlling agent; and at least one binder; wherein, the proportion of binder in the tablet is greater than about 3.0% of the total weight of the tablet and wherein, the pH modifier is present in a proportion of more than about 15 parts for each of the 100 parts of the desvenlafaxine base is also provided. Also provided is process for preparation of extended release tablet.

### FIELD OF THE INVENTION

The present invention relates to extended release pharmaceutical compositions comprising desvenlafaxine base and processes for the preparation thereof.

### BACKGROUND OF THE INVENTION

Depression is a psychiatric disorder that can be a primary condition or may co-exist with other mental, psychiatric or physical illnesses. Many subtypes of depression have been described, including major depressive disorder, bipolar disorder, dysthymic disorder, adjustment disorder, seasonal affective disorder, premenstrual dysphoric disorder, postpartum depression, psychotic depression and atypical depression.

O-desmethylvenlafaxine or desvenlafaxine (ODV) are adopted names for the drug compound having a chemical name RS-4-[2-dimethylamino-1-(1-hydroxycyclohexyl)ethyl]phenol. Its marketed succinate salt is represented by structural Formula I.

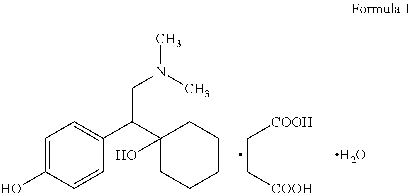

Desvenlafaxine is prescribed for treating major depressive disorders. Desvenlafaxine is the major metabolite of venlafaxine that selectively blocks the reuptake of serotonin and norepinephrine and is currently marketed in the USA as PRISTIQ® in the form of extended release tablets containing desvenlafaxine succinate equivalent to 50 mg and 100 mg of desvenlafaxine, for once-a-day oral administration.

Desvenlafaxine base, chemically named 1[2-(dimethylamino)-1-(4-phenol)ethyl]-cyclohexanol, is disclosed as various pharmaceutically acceptable salts and exemplified as fumarate salt with m.p. in the range of 140-142° C. in U.S. Pat. No. 4,535,186. U.S. Pat. No. 6,673,838 has disclosed various synthetic techniques for preparation of the desvenlafaxine base. However, it prefers the selection of salts of desvenlafaxine base over the desvenlafaxine base itself to prepare the solid dosage forms of its embodiments. Further, It even discriminates against the use of the fumarate salt of desvenlafaxine due to unsuitable physicochemical and permeability characteristics. Thus it teaches the reader away from the use of desvenlafaxine base per se to prepare solid oral dosage forms and additionally adds specific emphasis to the use of succinate salt of desvenlafaxine base. U.S. Pat. No. 6,673,838 teaches formulations of desvenlafaxine succinate in the form of capsules and tablets without the use of any specific pharmaceutically acceptable acid as an excipient and attaches importance to the extended release formulations of desvenlafaxine succinate that help to reduce adverse effects such as nausea, vomiting and diarrhea. The extended release tablet of desvenlafaxine succinate as in U.S. Pat. No. 6,673,838 is prepared using hydroxypropyl methylcellulose (HPMC 2208 USP 100, 100 SR) as a matrix.

A dosage form of desvenlafaxine base per se is not available in the market. It would be highly desirable to eliminate the step of having to convert desvenlafaxine base into a salt for use in the solid dosage form. The present invention provides for extended release formulations of desvenlafaxine base having excellent dissolution properties.

Further Indian application 355/MUM/2009 discusses once daily extended release formulations for desvenlafaxine. However, the reproducibility of the examples of the specification was found to be poor in terms of dissolution data, weight variation beyond acceptable limits and undesired granulation characteristics leading to unevenly sized granules. There are also problems of having residual organic solvents in the formulations. Thus, they were not suitable for scale-up to prepare a larger batch of formulation. The particle size distribution of the desvenlafaxine base performed in duplicate used for the examples of this application is as follows—

| | | | | Specific |
|---|---|---|---|---|
| | % of particles | | | Surface |
| | <=10 | <=50 | <=90 | area m²/g |
| Particle size | 28.611 | 78.563 | 185.210 | 0.124 |
| in microns | 27.825 | 77.837 | 185.000 | 0.126 |

To tackle these problems, the present invention provides some of the solutions such as usage of a particular particle size of desvenlafaxine base, usage of a specific proportion of different excipients in the tablets, usage of a higher percentage of binder in the tablets etc. Also, the present invention helps to avoid the chances of having residual organic solvents in the tablets.

Also as per some of the embodiments of our invention, it is possible to make formulations of desvenlafaxine base using convenient and simple processes that exhibit a reproducible dissolution profile or bioequivalence similar to PRISTIQ®.

### SUMMARY OF THE INVENTION

In one general aspect, there is provided an extended release pharmaceutical composition comprising:
a) micronized desvenlafaxine base,
b) at least one pH modifier and
c) at least one release controlling agent. In yet another aspect, there is provided a process for preparing the extended release pharmaceutical composition comprising the steps of:
a) mixing micronized desvenlafaxine base, at least one pH modifier and one or more release controlling agents and
b) granulating the mixture of step (a) with or without a binder solution.

In another aspect, there is provided an extended release tablet comprising:
a) micronized desvenlafaxine base,
b) at least one pH modifier and
c) at least one release controlling agent.

In another aspect, there is provided a process for preparing the extended release tablet comprising the steps of:
a) mixing micronized desvenlafaxine base, at least one pH modifier and one or more release controlling agents;
b) granulating the mixture of step (a) with or without a binder solution, to form granules;
c) mixing the granules with at least one pharmaceutically acceptable excipient and/or with at least one release controlling agent, to form a blend;
d) compressing the blend to form the tablet and
e) optionally coating the tablet.

In another aspect, there is provided an extended release monolithic tablet comprising:
a) micronized desvenlafaxine base,
b) at least one pH modifier and
c) at least one release controlling agent present intragranularly and at least one release controlling agent present extragranularly.

In another aspect, there is provided a process for preparing the extended release monolithic tablet comprising the steps of:
a) mixing micronized desvenlafaxine base, at least one pH modifier and one or more release controlling agents;
b) granulating the mixture of step (a) with or without a binder solution, to form granules;
c) mixing the granules with at least one pharmaceutically acceptable excipient and at least one release controlling agent to form a blend;
d) compressing the blend to form the tablet and
e) optionally coating the tablet.

In one general aspect, there is provided an extended release tablet comprising:
(a) micronized desvenlafaxine base;
(b) at least one pH modifier;
(c) at least one release controlling agent and
(d) at least one binder
   wherein, the proportion of binder in the tablet is greater than about 3.0% of the total weight of the tablet and wherein, the pH modifier is present in a proportion of more than about 15 parts for each of the 100 parts of the desvenlafaxine base.

In yet another aspect, there is provided an extended release tablet comprising:
(a) micronized desvenlafaxine base;
(b) at least one pH modifier;
(c) at least one release controlling agent present both intragranularly as well as extragranularly and
(d) at least one binder
   wherein, the proportion of binder in the tablet is greater than about 3.0% of the total weight of the tablet and wherein, the pH modifier is present in a proportion of more than about 15 parts for each of the 100 parts of the desvenlafaxine base.

### DETAILED DESCRIPTION OF THE INVENTION

Generally provided herein; are extended release pharmaceutical compositions comprising desvenlafaxine base and processes to prepare the same.

The extended release pharmaceutical compositions (in suitable dosage and dosage regimens) of the present invention can be used for treating patients suffering from agoraphobia, anorexia nervosa, anxiety, attention deficit disorder, autism, bipolar disorder, borderline personality disorder, bulimia nervosa, central pain, chronic back pain, chronic fatigue syndrome, cocaine and alcohol addiction, depression, dysthymia, epilepsy, fibromyalgia, generalized anxiety disorder, gilles de la tourette syndrome, major depressive disorder, migraine, neuropathic pain such as diabetic neuropathy, obesity, obsessive compulsive disorder, pain, panic disorder, parkinson's disease, phantom limb pain, post traumatic stress disorder, postherpetic neuropathy, premature ejaculation, premenstrual dysphoric disorder, raynaud's syndrome, schizophrenia, sexual dysfunction, shy drager syndrome, social anxiety disorder, urinary incontinence and vasomotor flushing. The extended release pharmaceutical compositions (in suitable dosage and dosage regimens) of the present invention can also be administered to prevent relapse or recurrence of depression, to induce cognitive enhancement, to treat cognitive impairment, and in regimens for cessation of smoking or other tobacco uses to treat hypothalamic amenorrhea in depressed and non-depressed human females.

The term “extended release pharmaceutical composition” as used herein, should be understood in contrast to an immediate release pharmaceutical composition; indicating that, the formulation does not release the full content of the active ingredient immediately after oral dosing and the formulation allows a reduction in dosage frequency to a human subject in comparison to an immediate release pharmaceutical composition.

The term “extended release tablet” as used herein, should be understood in contrast to an immediate release tablet; indicating that, the tablet does not release the full content of the active ingredient immediately after oral dosing and thus allows a reduction in dosage frequency to a human subject in comparison to an immediate release tablet.

The term “micronized” as used herein means a particle size of desvenlafaxine base containing particles prepared by any process or methods of particle size reduction. For example, the suitable particle sizes of the desvenlafaxine base containing particles of the present invention can be obtained by any milling, grinding, micronizing or other particle size reduction method known in the art. For example, the particles of desvenlafaxine base having a d₉₀ of more than 3 microns, more than 7 microns, more than 10 microns, more than 50 microns, more than 100 microns or more than 200 microns can be used to prepare the pharmaceutical compositions of the invention, as long as the desirable drug release characteristics of the pharmaceutical compositions vested in the spirit of the present invention are preserved. Preferably, the particles of desvenlafaxine base having a d₉₀ of less than 3 microns, less than 7 microns, less than 10 microns, less than 50 microns, less than 100 microns, less than 175 microns or less than 200 microns can be used to prepare the pharmaceutical compositions of the invention.

The specific surface area of the desvenlafaxine base can be greater than 0.1 m²/g, preferably greater than 0.2 m²/g, more preferably, greater than 2.5 m²/g and most preferably, between 2.5-3.5 m²/g. For example, the following micronized desvenlafaxine particle size distributions as per following table, demonstrates the particle size that can be suitably used to prepare the desvenlafaxine tablets.

### Particle Size Distribution For Desvenlafaxine Base Particles

| | | | | Specific |
|---|---|---|---|---|
| | % of particles | | | Surface |
| | <=10 | <=50 | <=90 | area m²/g |
| Particle size | 0.969 | 4.138 | 11.322 | 2.65 |
| in microns | 1.006 | 4.177 | 11.361 | 2.64 |
| | 1.01 | 4.297 | 12.767 | 2.55 |
| | 0.862 | 3.593 | 9.237 | 3.01 |
| | 0.889 | 3.639 | 9.664 | 2.93 |
| | 0.918 | 3.793 | 10.448 | 2.82 |
| | 0.91 | 3.965 | 9.667 | 2.83 |
| | 0.876 | 3.801 | 9.49 | 2.93 |
| | 0.866 | 3.781 | 9.506 | 2.95 |
| | 0.914 | 3.977 | 9.668 | 2.82 |
| | 0.898 | 3.861 | 9.415 | 2.88 |
| | 0.877 | 3.782 | 9.427 | 2.93 |

“Specific surface area” is the ratio of the surface area of desvenlafaxine base particles to its unit mass; expressed herein in m²/g.

The pharmaceutical compositions of the present invention contain, desvenlafaxine base in an amount of about 5-50% w/w, preferably 10-40% w/w of the total weight of the pharmaceutical composition.

Unless otherwise indicated, “% w/w” as used herein, means percentage weight with respect to the total weight of the pharmaceutical composition.

As used herein the term “pH modifier” refers to a pharmaceutically acceptable organic or inorganic acid substance. Examples thereof include but are not limited to a carbomer, acid anhydride, alginic acid, a latent acid such as glucono-d-lactone, organic acids that contain one or more acidic groups, preferably compounds containing acidic groups selected from carboxylic and sulfonic acid groups, more preferably those which are solid at ambient temperature, and most preferably those which have 2 or more acidic groups, mono, di- or polybasic carboxylic acids and mono, di or tri-sulfonic acids such as—sorbic acid adipic acid, malonic acid, glutaric acid, maleic acid or fumaric acid. Other examples include water-soluble aryl carboxylic acids containing up to 20 carbon atoms or substituted carboxylic acids, for example—hydroxy substituted monocarboxylic acids such as gluconic acid, solid forms of lactic acid, glycolic acid or ascorbic acid; hydroxy substituted dicarboxylic acids such as malic acid, tartaric acid, tartronic acid or mucic acid; tri-carboxylic acids, for example citric acid; or amino acids with an acidic side chain, such as glutamic acid or aspartic acid. A pH modifier is employed in the embodiments of the present invention to shift the pH within and in the vicinity of the desvenlafaxine base formulation to more acidic conditions. The use of solid acids or pharmaceutical acceptable salts thereof as pH modifiers is particularly convenient for the manufacture of compositions according to the embodiments of the present invention.

The pH modifier is preferably selected from fumaric acid, aspartic acid, glutamic acid, adipic acid, cinnamic acid, ascorbic acid, ascorbyl palmitate, citric acid, malic acid, tartaric acid, L-lactic acid, maleic acid, oxalic acid, stearic acid, orotic acid, sebacic acid or mixtures thereof. Citric acid is the pH modifier that is the most preferable to prepare the extended release tablets of desvenlafaxine base.

The composition is therefore, preferably adapted such that its pH within and in the vicinity of the desvenlafaxine base composition is substantially maintained in at least a section of the gastrointestinal tract. The quantity and properties of the pH modifier should therefore, be tailored to optimize absorption of the desvenlafaxine base. This may involve the use of a pH modifier having a dissolution rate substantially similar to that of the desvenlafaxine base and/or a sufficient quantity of pH modifier to maintain the pH within and in the vicinity of the desvenlafaxine base composition. The pH modifier may be present in an amount from about 1-25% w/w, preferably from about 1-20% w/w of the total weight of the pharmaceutical composition.

Release controlling agents are used in the pharmaceutical compositions to control the rate of release of desvenlafaxine base from the composition and include water soluble/swellable polymers or mixtures thereof. The release controlling agent may be present in an amount from about 1-40% w/w, preferably from about 5-30% w/w of the total weight of the pharmaceutical composition. The release controlling agents can be present intragranularly and/or extragranularly when granules of desvenlafaxine base are prepared to be incorporated into a tablet. Preferably, the release controlling agents are used both intragranularly and extragranularly. When used intragranularly, the proportion of the binder and the release controlling agent is so adjusted that not more than about 5 parts of release controlling agent is present intragranularly for each part of binder, preferably, not more than 4 parts of release controlling agent is present intragranularly for each part of binder.

Examples of water soluble polymers include, but are not limited to, one or more of cellulose derivatives, gums, vinyl alcohol or vinylpyrrolidone-based polymers or mixtures thereof. The cellulose derivatives may include one or more of hydroxypropyl methylcellulose, hydroxyethyl cellulose, hydroxypropyl cellulose, carboxymethyl cellulose, sodium carboxymethyl cellulose or mixtures thereof. The gums may include one or more of xanthan gum, karaya gum, locust bean gum, alginic acid, sodium alginate or mixtures thereof. The vinyl alcohol or vinylpyrrolidone-based polymers may include one or more of polyvinyl alcohol, polyvinylpyrrolidone or mixtures thereof.

The term “pharmaceutically acceptable excipients,” as used herein, includes all excipients used in the art of manufacturing solid dosage forms. Examples of pharmaceutically acceptable excipients include binders, diluents, surfactants, lubricants/glidants, coloring agents and the like.

Suitable binders include, for example, methyl cellulose, hydroxypropyl cellulose, hydroxypropyl methylcellulose, polyvinylpyrrolidone, gelatin, gum arabic, ethyl cellulose, polyvinyl alcohol, pregelatinized starch, agar, tragacanth, sodium alginate, propylene glycol and the like or mixtures thereof.

Suitable diluents include, for example, calcium carbonate, calcium phosphate-dibasic, calcium phosphate-tribasic, calcium sulfate, microcrystalline cellulose, dextrates, dextrins, dextrose excipients, fructose, kaolin, lactitol, lactose, mannitol, sorbitol, starch, starch pregelatinized, sucrose, compressible sugars and the like or mixtures thereof.

Suitable surfactants include, for example, both non-ionic and ionic (cationic, anionic and zwitterionic) surfactants such as sodium lauryl sulfate, poloxamers (copolymers of polyoxyethylene and polyoxypropylene), natural or synthetic lecitins, sorbitan esters, polyoxyethylene sorbitan fatty acid esters, polyoxyethylene castor oil derivatives, polyoxyethylene stearates or mixtures thereof.

Suitable lubricants/glidants include, for example, colloidal silicon dioxide, stearic acid, magnesium stearate, calcium stearate, talc, hydrogenated castor oil, sucrose esters of fatty acids, microcrystalline wax, yellow beeswax, white beeswax and the like.

Coloring agents include any FDA approved colors for oral use.

The extended release pharmaceutical compositions may further comprise one or more non-functional coatings and/or one or more release controlling coatings.

Non-functional coating is a coating that does not affect the release rate of the desvenlafaxine base. Non-functional coatings can facilitate in forming a smooth surface and better appearance of the pharmaceutical compositions. Non-functional coatings can also help in overcoming common problems, including fragmentation of the coated units due to mechanical stress generated during compression of coated units into tablets or filling into capsules/sachets. Examples of substances suitable for a non-functional coat include hydroxypropyl cellulose, hydroxypropyl methylcellulose (hypromellose) or polyvinyl alcohol. In certain embodiments, the non-functional coating is an Opadry® coating, which includes hydroxypropyl methylcellulose and polyethylene glycol as key ingredients.

The release controlling coatings are useful to modulate the release of desvenlafaxine base from the pharmaceutical compositions. Examples include coatings containing hydrophobic polymeric substances such as for example, ethyl cellulose, methacrylic acid polymers and copolymers, fatty acids and esters thereof, waxes, high molecular weight fatty alcohols and the like used in suitable amount as known to a person skilled in the art. The hydrophobic polymeric substances may be present in an amount ranging from about 0.05-20% w/w, more particularly in an amount ranging from about 0.5-10% w/w of the total weight of the pharmaceutical composition.

Coating solutions may be applied using techniques, for example, spray coating in a conventional coating pan or fluidized bed processor or dip coating. Solutions or dispersions of polymers can be prepared in solvents, for example, dichloromethane, isopropyl alcohol, acetone, methanol, ethanol, water or mixtures thereof. Coating solutions may further comprise other pharmaceutically acceptable ingredients, for example, plasticizers, coloring agents and surfactants.

Solvents such as water, methanol, ethanol, isopropyl alcohol, acetone, methylene chloride and the like or their mixtures can be used wherever necessary to prepare the compositions of the invention for purposes such as granulation, coating and the like.

The extended release pharmaceutical compositions may be formulated as granules filled into hard gelatin capsules or sachets, or formed into tablets or mini-tablets that can be filled into capsules.

The general procedure of manufacturing the extended release pharmaceutical composition of desvenlafaxine base is as follows:
a) mixing micronized desvenlafaxine base, at least one pH modifier and one or more release controlling agents and
b) granulating the mixture of step (a) with or without a binder solution.

Dry granulation techniques comprise mixing the desvenlafaxine base with pH modifier, release controlling agent/s and optionally one or more excipients (except lubricants), compacting the mixture in a compactor (e. g. a roller compactor), or double compression, milling the compacted mass, screening the milled granules.

Wet granulation techniques comprise mixing desvenlafaxine base, pH modifier, release controlling agent/s and optionally one or more excipients, granulating the blend using either solution of a binder or solvent alone, drying the granules. Solvents or mixtures of solvents like isopropyl alcohol or purified water are suitable for wet granulation.

Suitable equipment such as sifter, planetary mixer, jacketed or conventional rapid mixer granulator, roll compactor, milling equipment like oscillatory granulator and jet mill, extruder/spheronizer, fluid bed processor with top and/or bottom spray facilities, fluid bed dryer, spray dryer are selectively used to prepare the granules by wet or dry granulation processes in the manner known to a person skilled in the art and also, references such as Remington's Pharmaceutical Sciences, 18^{th} edition, 1990, Mack publishing Company Easton, Pa. 18042 (and even the recent editions) which describe such processes for the preparation of solid dosage forms are well known in the art.

The granules so obtained optionally may further be mixed with suitable pharmaceutically acceptable excipients or additional release controlling agents as per the dosage form and release profile desired using techniques known to a person skilled in the art. Lubrication of the granules depends on the desired flow properties of the granules and is optional. The granules may be optionally coated with release controlling and/or non-functional coatings as described above. The granules can be filled into hard gelatin capsules or sachets, or formed into tablets or mini-tablets that can be filled into capsules.

According to an embodiment of the invention; a process for preparing the extended release tablet comprises the steps of:
a) mixing micronized desvenlafaxine base, at least one pH modifier and one or more release controlling agents;
b) granulating the mixture of step (a) with or without a binder solution, to form granules;
c) mixing the granules with at least one pharmaceutically acceptable excipient and/or with at least one release controlling agent, to form a blend;
d) compressing the blend to form the tablet and
e) optionally coating the tablet.

According to another variation of the above embodiment of the invention; a process for preparing the extended release monolithic tablet comprises the steps of:
a) mixing micronized desvenlafaxine base, at least one pH modifier and one or more release controlling agents;
b) granulating the mixture of step (a) with or without a binder solution, to form granules;
c) mixing the granules with at least one pharmaceutically acceptable excipient and at least one release controlling agent to form a blend;
d) compressing the blend to form the tablet and
e) optionally coating the tablet.

The tooling of the tablet punching machine required for the tablets is based on the dimensions and design of the tablets and can be varied, as per requirements to prepare the tablets of various shapes and sizes.

The tablets are optionally coated with release controlling and/or non-functional coating as described above.

While the present invention has been described in terms of its specific embodiments, certain modifications and equivalents will be apparent to those skilled in the art and are included within the scope of the present invention.

The extended release properties of the compositions of the present invention may be demonstrated by monitoring the dissolution of the desvenlafaxine base pharmaceutical compositions. The dissolution of the desvenlafaxine base may be monitored using standard procedures well known to those skilled in the art. For example, the dissolution test procedures, such as the rotating basket method or paddle method or reciprocating cylinder or flow-through cell, disclosed in the U.S. Pharmacopeia or British Pharmacopeia can be judiciously used. Such procedures include those in which the formulation is immersed in a suitable medium, for example an aqueous medium such as water, 0.9% NaCl in water or hydrochloric acid and aliquots of the medium are withdrawn at various time points over a period of 24 hours. The aliquots are analyzed using high pressure liquid chromatography (HPLC) with UV detection to determine the concentration of released desvenlafaxine base using standard methodology. In a particular embodiment, dissolution tests were conducted on the extended release desvenlafaxine base tablets as obtained by the procedure described in the examples 1 to 3 and 6 to 7.

For comparison purposes, it can be observed that extended release desvenlafaxine base tablets as per the exact formula of Example 3 (without the pH modifier) did not exhibit equivalent dissolution profile to that of PRISTIQ®. The results as in Table 4 and 6 illustrate that, the use of pH modifier as per the formulae of Examples 1, 2 and 4 help in achieving extended release desvenlafaxine base tablets which exhibit an equivalent dissolution profile to that of PRISTIQ®

For comparison purposes, it can be observed that extended release desvenlafaxine base tablets Examples 6 and 7 exhibit an equivalent dissolution profile to that of PRISTIQ®

The compositions according to the various embodiment of the invention may be formulated to produce formulations that are bioequivalent to PRISTIQ®. A representative composition prepared according to the Example 4, as illustrated below shows the pharmacokinetic profile as in Table 1.

A further representative desvenlafaxine base tablets prepared according to the Example 6, as illustrated below shows the pharmacokinetic profile as in Table 2.

A randomized, two treatment, two sequence, two period, single dose, crossover biostudy was performed on extended release desvenlafaxine base 100 mg tablets of Example 4 versus PRISTIQ® 100 mg under fed conditions on 13 healthy human male volunteers. As shown in Table 1, the 100 mg compositions of desvenlafaxine base prepared according to the formulation of Example 4 were found to be bioequivalent to the reference product, 100 mg PRISTIQ® Further a randomized, two treatment, two sequence, two period, single dose, crossover biostudy was performed on extended release desvenlafaxine base 100 mg tablets of Example 6 versus PRISTIQ® 100 mg under fasting conditions on 13 healthy human male volunteers. As shown in Table 2, the 100 mg tablets of desvenlafaxine base prepared according to the formulation of Example 6 were found to be bioequivalent to the reference product, 100 mg PRISTIQ® The biostudy was performed as per the methods known to a person skilled in the art. “Guidance for Industry Bioavailability and Bioequivalence Studies for Orally Administered Drug Products—General Considerations by U.S. Department of Health and Human Services, Food and Drug Administration, Center for Drug Evaluation and Research (CDER), March 2003, BP, Revision 1” is one such document that provides the general information about biostudies. The pharmacokinetic analysis was carried out using WinNolin and SAS® software. A summary of the study results is presented below.

TABLE 1
Summary statistics of Test (T): Desvenlafaxine base extended
release tablets 100 mg of example 4 Vs. Reference (R):
PRISTIQ ® (desvenlafaxine succinate extended
release tablets 100 mg) in healthy adult human male subjects
(N = 13) under fed conditions.

| | Cₘₐₓ | AUC₀₋ₜ | AUC_{0-INF} |
|---|---|---|---|
| PK Parameters | (ng/mL) | (ng · hr/mL) | (ng · hr/mL) |
| N | | | |
| T | 13 | 13 | 13 |
| R | 13 | 13 | 13 |
| Geometric Least square mean | | | |
| T | 326.384 | 6635.915 | 6739.024 |
| R | 315.862 | 6670.743 | 6782.474 |
| Ratio (T/R) | 103.33 | 99.48 | 99.36 |
| 90% C.I. (T Vs. R) | | | |
| LCL | 95.81 | 89.92 | 90.17 |
| UCL | 111.44 | 110.05 | 109.49 |

TABLE 2
Summary statistics of Test (T): Desvenlafaxine base extended
release tablets 100 mg of example 6 Vs. Reference (R):
PRISTIQ ® (desvenlafaxine succinate extended
release tablets 100 mg) in healthy adult human male subjects
(N = 13) under fasting conditions.

| | Cₘₐₓ | AUC₀₋ₜ | AUC_{0-INF} |
|---|---|---|---|
| PK Parameters | (ng/mL) | (ng · hr/mL) | (ng · hr/mL) |
| N | | | |
| T | 13 | 13 | 13 |
| R | 13 | 13 | 13 |
| Geometric Least square mean | | | |
| T | 267.4086 | 6072.3233 | 6161.7058 |
| R | 241.3637 | 5489.6073 | 5580.1543 |
| Ratio (T/R) | 110.79 | 110.61 | 110.42 |
| 90% C.I. (T Vs. R) | | | |
| LCL | 98.23 | 99.91 | 99.96 |
| UCL | 124.95 | 122.47 | 121.98 |

Note-
The statistical analysis was done on the log transformed values; the antilog of the mean is reported.
T—Test (Desvenlafaxine base extended release Tablets 100 mg) of Example 4 and Example 6 respectively
R—Reference [PRISTIQ ® (Desvenlafaxine Succinate extended release Tablets 100 mg)]
LCL—Lower Clearance level
UCL—Upper Clearance level
AUC₀₋ₜ—Area under the plasma/serum/blood concentration-time curve from time zero to time t, where t is the last time point with measurable concentration for individual formulation.
AUC_{0-INF}—Area under the plasma/serum/blood concentration-time curve from time zero to time infinity, where AUC_{0-INF} = AUC₀₋ₜ + Ct/λ z, Ct is the last measurable drug concentration and λ z is the terminal or elimination rate constant calculated according to an appropriate method.

The ratio of least-squares means and 90% confidence intervals derived from the analysis of the log transformed parameters Cₘₐₓ, AUC ₀₋ₜ and AUC_{0-INF} were within the 80-125% range for T vs. R comparisons

The examples mentioned below, demonstrate some illustrative procedures for preparing the extended release pharmaceutical composition as described herein.

The examples are provided to illustrate particular aspects of the disclosure and do not limit the scope of the present invention as defined by the claims.

### Examples 1-3

TABLE 3
Quantitative compositions of Desvenlafaxine base per tablet

| | | Example 1 | | Example 2 | | Example 3 | |
|---|---|---|---|---|---|---|---|
| No. | Ingredients | mg | % w/w | mg | % w/w | mg | % w/w |
| 1 | Desvenlafaxine base | 100.85 | 28.81 | 50.42 | 14.41 | 50.31 | 29.59 |
| 2 | Alginic acid | 10.00 | 2.86 | 5.00 | 1.43 | — | — |
| 3 | Citric acid | 15.00 | 4.29 | 10.00 | 2.86 | — | — |
| | monohydrate | | | | | | |
| 4 | Methocel K100M CR | 13.00 | 3.71 | 53.00 | 15.14 | 50 | 29.41 |
| | [hypromellose] | | | | | | |
| 5 | Microcrystalline | 136.15 | 38.90 | 156.58 | 44.74 | 47.69 | 28.05 |
| | cellulose (Avicel PH | | | | | | |
| | 101) | | | | | | |
| 6 | PVPK-30 | 10.00 | 2.86 | 10.00 | 2.86 | 5 | 2.94 |
| | [polyvinylpyrrolidone] | | | | | | |
| 7 | Isopropyl alcohol | q.s. | — | q.s. | — | q.s. | — |
| 8 | Purified Water | q.s. | — | q.s. | — | q.s. | — |
| 9 | Methocel K100M CR | 62.00 | 17.71 | 62.00 | 17.71 | 15 | 8.82 |
| | [hypromellose] | | | | | | |
| 10 | Talc | 2.00 | 0.57 | 2.00 | 0.57 | 1 | 0.59 |
| 11 | Magnesium Stearate | 1.00 | 0.29 | 1.00 | 0.29 | 1 | 0.59 |
| | Total weight of each | 350 | | 350 | | 170 | |
| | tablet (mg) | | | | | | |

q.s. = quantity sufficient

### Process:

Desvenlafaxine base, alginic acid, citric acid monohydrate, Methocel K100M CR and microcrystalline cellulose were weighed and sifted, mixed to form a blend and transferred to rapid mixer granulator. PVPK-30 was dissolved in the mixture of isopropyl alcohol and purified water and used for granulation of the blend. After granulation the wet mass was sifted and dried. After drying, dried granules were sifted and mixed with Methocel K100M CR, talc and magnesium stearate and properly mixed to form a lubricated blend.

The lubricated blend was subjected to compression on rotary tablet compression machine by using suitable tooling. The tablet prepared by the above process was further coated with opadry (key ingredients—hydroxypropylmethylcellulose, polyethylene glycol, titanium dioxide, talc) with a 3% increase in tablet weight.

### Dissolution Studies Data:

### Conditions

Media: 900 ml 0.9% NaCl in water
Apparatus: USP type I (basket)
Temperature: 37±0.5° C.
RPM: 100

TABLE 4
DISSOLUTION PROFILE COMPARISON WITH PRISTIQ ®

| | % Drug Release | | | |
|---|---|---|---|---|
| Time (hrs) | PRISTIQ ® | Example 1 | Example 2 | Example 3 |
| 1 | 15 | 17 | 19 | 2 |
| 2 | 24 | 25 | 28 | 3 |
| 4 | 37 | 37 | 43 | 7 |
| 8 | 58 | 59 | 62 | 15 |
| 12 | 71 | 72 | 75 | 25 |
| 24 | 89 | 87 | 90 | 53 |
| F2 | — | 87 | 69 | 24 |

Based on the above similarity factor (F₂ values), which need be greater than 50%, it was seen that the F₂ value obtained for Example 3 was below average.

### Example 4

TABLE 5
QUANTITATIVE COMPOSITION
OF DESVENLAFAXINE BASE

| | | Quantity | % |
|---|---|---|---|
| No. | Ingredients | (mg/tablet) | w/w |
| 1 | Desvenlafaxine base | 100.00 | 27.74 |
| 2 | Alginic acid USP | 10.00 | 2.77 |
| 3 | Citric Acid monohydrate USP | 15.00 | 4.16 |
| 4 | Methocel K100M CR USP [hypromellose] | 13.00 | 3.61 |
| 5 | Micro-crystalline cellulose (Avicel PH 101) | 129.50 | 35.92 |
| | NF | | |
| 6 | PVPK- 30 [polyvinylpyrrolidone] | 15.00 | 4.16 |
| 7 | Isopropyl alcohol USP | q.s. | q.s. |
| 8 | Purified Water USP | q.s. | q.s. |
| 9 | Methocel K100M CR USP [hypromellose] | 62.00 | 17.20 |
| 10 | Talc USP | 2.00 | 0.56 |
| 11 | Magnesium Stearate USP | 3.50 | 0.97 |
| 12 | Opadry | 10.5 | 2.91 |

q.s. = quantity sufficient
Weight of compressed tablet—350 mg
Weight of Film coated tablet—360.5 mg (3% increase in tablet weight)

Process—

Desvenlafaxine base, alginic acid, citric acid monohydrate, methocel K100M CR and microcrystalline cellulose were weighed and sifted, mixed to form a blend and transferred to a rapid mixer granulator. PVPK-30 was dissolved in the mixture of isopropyl alcohol and purified water and used for granulation of the blend. After granulation the wet mass was sifted and dried. after drying, dried granules were sifted and mixed with sifted methocel K100M CR, talc and magnesium stearate and properly mixed to form a lubricated blend. The lubricated blend was subjected to compression on a rotary tablet compression machine by using suitable tooling. The compressed tablets were then coated with aqueous solution of opadry [key ingredients—hydroxypropylmethylcellulose, polyethylene glycol, titanium dioxide, talc] film coating material.

### Dissolution Studies Data:

### A) Conditions—

Media: 900 ml acetate buffer pH =4.5
Apparatus: USP type I (basket)
Temperature: 37±0.5° C.
RPM: 100

### B) Conditions—

Media: 900 ml 0.1 N HCl
Apparatus: USP type I (basket)
Temperature: 37±0.5° C.
RPM: 100

TABLE 6
DISSOLUTION PROFILE COMPARISON WITH PRISTIQ ®

| | % Drug Release | | | |
|---|---|---|---|---|
| | PRISTIQ ® | | PRISTIQ ® | |
| | [100 mg] | Example 4 | [100 mg] | Example 4 |
| Time (hrs) | Acetate buffer pH = 4.5 | | 0.1N HCl | |
| 1 | 19 | 19 | 25 | 32 |
| 2 | 29 | 30 | 37 | 44 |
| 4 | 45 | 46 | 56 | 66 |
| 8 | 66 | 68 | 80 | 90 |
| 12 | 81 | 89 | 94 | 102 |
| 16 | 92 | 100 | 102 | 106 |
| 20 | 97 | 107 | 105 | 107 |
| 24 | 99 | 109 | 107 | 109 |

| | | |
|---|---|---|
| F2 | 57.81 | 57.59 |

Example 5

TABLE 7
QUANTITATIVE COMPOSITION
OF DESVENLAFAXINE BASE

| | | Quantity | % |
|---|---|---|---|
| No. | Ingredients | (mg/tablet) | w/w |
| 1 | Desvenlafaxine base | 50.00 | 13.87 |
| 2 | Alginic acid USP | 5.00 | 1.39 |
| 3 | Citric Acid monohydrate USP | 10.00 | 2.77 |
| 4 | Methocel K100M CR USP [hypromellose] | 53.00 | 14.70 |
| 5 | Microcrystalline cellulose (Avicel PH 101) | 149.50 | 41.47 |
| | NF | | |
| 6 | PVPK- 30 [polyvinylpyrrolidone] | 15.00 | 4.16 |
| 7 | Isopropyl alcohol USP | q.s. | q.s. |
| 8 | Purified Water USP | q.s. | q.s. |
| 9 | Methocel K100M CR USP [hypromellose] | 62.00 | 17.20 |
| 10 | Talc USP | 2.00 | 0.56 |
| 11 | Magnesium Stearate USP | 3.50 | 0.97 |
| 12 | Opadry | 10.5 | 2.91 |

q.s. = quantity sufficient
Weight of compressed tablet—350 mg
Weight of Film coated tablet—360.5 mg (3% increase in tablet weight)

Process—

Desvenlafaxine base, alginic acid, citric acid monohydrate, methocel K100M CR and microcrystalline cellulose were weighed and sifted, mixed to form a blend and transferred to a rapid mixer granulator. PVPK-30 was dissolved in a mixture of isopropyl alcohol and purified water and used for granulation of the blend. After granulation the wet mass was sifted and dried. After drying, dried granules were sifted and mixed with sifted methocel K100M CR, talc and magnesium stearate and properly mixed to form a lubricated blend. The lubricated blend was subjected to compression on a rotary tablet compression machine by using suitable tooling. The compressed tablets were then coated with aqueous solution of opadry [key ingredients—hydroxypropylmethylcellulose, polyethylene glycol, titanium dioxide, talc] film coating material.

### Examples 6-7

Each of the examples were repeated in quadruple and were named as 6A,6B,6C,6D and 7A,7B,7C,7D

TABLE 8
Quantitative Composition of Desvenlafaxine
Base Tablets Example 6

| | | Quantity/ | % |
|---|---|---|---|
| No | Ingredients | tablet (mg) | w/w |
| Dry Mix | | | |
| 1 | Desvenlafaxine | 100.00 | 27.74 |
| 2 | Alginic Acid | 10.00 | 2.77 |
| 3 | Citric Acid Monohydrate Powder | 24.00 | 6.66 |
| 4 | Hypromellose | 30.00 | 8.32 |
| 5 | Microcrystalline Cellulose | 120.50 | 33.43 |
| Binder Solution | | | |
| 6 | Polyvinylpyrrolidone | 15.00 | 4.16 |
| 7 | Purified Water | q.s. | — |
| Extra granular | | | |
| 8 | Hypromellose | 45.00 | 12.48 |
| 9 | Talc | 2.00 | 0.56 |
| 10 | Magnesium Stearate | 3.50 | 0.97 |
| Core Tablet Weight | | 350.00 | |
| 11 | Opadry Brown 03F86990 | 10.50 | 2.91 |
| 12 | Purified Water | q.s. | — |

| | |
|---|---|
| Total Weight | 360.50 |

Opadry Brown 03F86990- key ingredients: hydroxypropyl methylcellulose, polyethylene glycol, titanium dioxide, talc

TABLE 9
Quantitative Composition of Desvenlafaxine
Base Tablets Example 7

| | | Quantity/ | % |
|---|---|---|---|
| No. | Ingredients | Tablet (mg) | w/w |
| Dry Mix | | | |
| 1 | Desvenlafaxine | 50.00 | 13.87 |
| 2 | Alginic Acid | 5.00 | 1.39 |
| 3 | Citric Acid Monohydrate Powder | 12.00 | 3.33 |
| 4 | Hypromellose | 53.00 | 14.70 |
| 5 | Microcrystalline Cellulose | 147.50 | 40.91 |
| Binder Solution | | | |
| 6 | Polyvinylpyrrolidone | 15.00 | 4.16 |
| 7 | Purified Water | q.s. | — |
| Extra granular | | | |
| 8 | Hypromellose | 62.00 | 17.20 |
| 9 | Talc | 2.00 | 0.56 |
| 10 | Magnesium Stearate | 3.50 | 0.97 |
| Core Tablet Weight | | 350.00 | |
| 11 | Opadry Pink 03F84770 | 10.50 | 2.91 |
| 12 | Purified Water | q.s. | — |

| | |
|---|---|
| Total Weight | 360.50 |

Opadry Pink 03F84770- key ingredients: hydroxypropyl methylcellulose, polyethylene glycol, titanium dioxide, talc

### Process for Examples 6 and 7:

1. Dispense Raw material quantities as mentioned in the formula.

2. Dissolve weighed quantity of Polyvinylpyrrolidone in Purified water under stirring in a stainless steel container to get clear solution.

3. Sift Desvenlafaxine, Alginic Acid, Citric Acid Monohydrate Powder, Hypromellose and Microcrystalline Cellulose through vibratory sifter equipped with sieve 30#. If required, mill 30# retain material through multi mill/turbo sifter cum multi mill equipped with 0.5 mm stainless steel screen at fast speed with knives forward and collect in in-process bulk container.

4. Load the mixture of Desvenlafaxine, Alginic Acid, Citric Acid Monohydrate Powder, Hypromellose and Microcrystalline Cellulose of step 3.0 to Rapid Mixer granulator and mix for 8 minutes at slow impeller speed. Granulate the blend in Rapid Mixer granulator by spraying the binder solution. Mix the wet mass for 1 minute. Unload the wet granular mass 5. Dry the wet mass of step 4.0 in Fluid bed equipment/Fluid Bed Dryer. Carry out the drying till Loss On Drying is achieved between 2.00%- 3.00% w/w at 105° C. on halogen moisture analyzer.

6. Pass the dried granules through sieve 20# equipped on vibratory sifter and collect the sifted material separately. Mill sieve 20# retained granules through multi mill/turbo sifter cum multi mill equipped with 1.0 mm stainless steel screen at fast speed with knives forward. Collect the granules.

7. Sift Hypromellose and Talc through sieve 40#.

Sift separately Magnesium Stearate through sieve 60#.

8. Add sifted Hypromellose and Talc with the sized granules of step 6.0 in a Conta blender and mix for 6 minutes at 12 rpm.

9. To blend of step 8.0 add sifted Magnesium Stearate of Step 7.0 and mix for 6 minutes at 12 rpm. Collect the lubricated granules.

10. Compress the lubricated blend of Step 9.0.

11. Coating solution preparation:

Transfer weighed Quantity of Purified water to a stainless steel container equipped with mechanical stirrer. Disperse Opadry Brown 03F86990 in purified water with continuous stirring and mix for 45 minutes. Filter through 200# sieve or nylon cloth.
Load the compressed tablets in to coating pan. Start coating and continue until 3.0% weight build up is achieved. Dry the coated tablets in the coating pan.

### Dissolution Studies Data—

TABLE 10
Comparative dissolution data of Desvenlafaxine base Extended-
Release Tablets 100 mg in 0.9% NaCl in water
Comparative dissolution data of Desvenlafaxine(base) Extended-Release
Tablets 100 mg in 0.9% NaCl in water, USP I, 100 RPM

| Time in hours | Pristiq ® | 6A | 6B | 6C | 6D |
|---|---|---|---|---|---|
| 1 | 18 | 17 | 17 | 17 | 17 |
| 2 | 27 | 26 | 27 | 27 | 27 |
| 4 | 41 | 42 | 43 | 43 | 43 |
| 8 | 62 | 66 | 68 | 67 | 66 |
| 12 | 76 | 86 | 84 | 84 | 84 |
| 16 | 87 | 98 | 94 | 94 | 94 |
| 20 | 94 | 102 | 98 | 101 | 101 |
| 24 | 98 | 106 | 100 | 102 | 102 |

| | | | | |
|---|---|---|---|---|
| F2 | 58.2 | 64.69 | 62.82 | 63.28 |
| F1 | 8.75 | 5.96 | 6.76 | 6.56 |

TABLE 11
Comparative dissolution data of Desvenlafaxine base Extended-
Release Tablets 50 mg in 0.9% NaCl in water
Comparative dissolution data of Desvenlafaxine(base) Extended-Release
Tablets 50 mg in 0.9% NaCl in water, USP I, 100 RPM

| Time in hours | Pristiq ® | 7A | 7B | 7C | 7D |
|---|---|---|---|---|---|
| 1 | 13 | 16 | 20 | 18 | 16 |
| 2 | 22 | 26 | 29 | 26 | 29 |
| 4 | 37 | 41 | 44 | 42 | 44 |
| 8 | 60 | 63 | 67 | 63 | 63 |
| 12 | 77 | 79 | 79 | 80 | 77 |
| 16 | 89 | 90 | 88 | 87 | 87 |
| 20 | 98 | 97 | 93 | 94 | 93 |
| 24 | 104 | 100 | 100 | 101 | 97 |

| | | | | |
|---|---|---|---|---|
| F2 | 75 | 62.63 | 70.51 | 63.55 |
| F1 | 4.4 | 8 | 5.8 | 6.8 |

1. An extended release pharmaceutical composition comprising:
a) micronized desvenlafaxine base,
b) at least one pH modifier and
c) at least one release controlling agent.

2. The extended release pharmaceutical composition according to claim 1, wherein the pH modifier is selected from the group consisting of an organic acid, inorganic acid, an acidic polymer, a latent acid or mixtures thereof

3. The extended release pharmaceutical composition according to claim 1, wherein the pH modifier is selected from the group consisting of fumaric acid, aspartic acid, glutamic acid, adipic acid, cinnamic acid, ascorbic acid, ascorbyl palmitate, citric acid, malic acid, tartaric acid, L-lactic acid, maleic acid, oxalic acid, stearic acid, orotic acid, sebacic acid or mixtures thereof

4. The extended release pharmaceutical composition according to claim 1, wherein the release controlling agent is selected from the group consisting of water soluble/swellable polymers or mixtures thereof

5. The extended release pharmaceutical composition according to claim 1, wherein the release controlling agent is selected from the group consisting of cellulose derivatives, gums, vinyl alcohol or vinylpyrrolidone-based polymers or mixtures thereof.

6. The extended release pharmaceutical composition according to claim 1, wherein the release controlling agent is selected from the group consisting of hydroxypropyl methylcellulose, hydroxyethyl cellulose, hydroxypropyl cellulose, carboxymethyl cellulose, sodium carboxymethyl cellulose, xanthan gum, karaya gum, locust bean gum, alginic acid, sodium alginate, polyvinyl alcohol, polyvinylpyrrolidone or mixtures thereof

7. A process for preparing the extended release pharmaceutical composition comprising the steps of:
a) mixing micronized desvenlafaxine base, at least one pH modifier and one or more release controlling agents and
b) granulating the mixture of step (a) with or without a binder solution.

8. An extended release tablet comprising:
a) micronized desvenlafaxine base,
b) at least one pH modifier and
c) at least one release controlling agent.

9. An extended release monolithic tablet comprising:
a) micronized desvenlafaxine base,
b) at least one pH modifier and
c) at least one release controlling agent present intragranularly and at least one release controlling agent present extragranularly.

10. An extended release tablet comprising:
(a) micronized desvenlafaxine base;
(b) at least one pH modifier;
(c) at least one release controlling agent; and
(d) at least one binder;
wherein, the proportion of binder in the tablet is greater than about 3.0% of the total weight of the tablet and wherein, the pH modifier is present in a proportion of more than about 15 parts for each of the 100 parts of the desvenlafaxine base.

11. The extended release tablet according to claim 10, wherein the specific surface area of the desvenlafaxine base particles is from about 2.5-3.5 m²/g.

12. An extended release tablet comprising:
(a) micronized desvenlafaxine base;
(b) at least one pH modifier;
(c) at least one release controlling agent present both intragranularly as well as extragranularly and
(d) at least one binder
wherein, the proportion of binder in the tablet is greater than about 3.0% of the total weight of the tablet and wherein, the pH modifier is present in a proportion of more than about 15 parts for each of the 100 parts of the desvenlafaxine base.

13. The extended release tablet according to claim 12, wherein the ratio of release controlling agent present intragranularly to the binder is less than about 5.25:1

14. The extended release tablet according to claim 8, wherein the pH modifier is selected from the group consisting of an organic acid, inorganic acid, an acidic polymer, a latent acid or mixtures thereof.

15. The extended release tablet according claim 14, wherein the pH modifier is selected from the group consisting of fumaric acid, aspartic acid, glutamic acid, adipic acid, cinnamic acid, ascorbic acid, ascorbyl palmitate, citric acid, malic acid, tartaric acid, L-lactic acid, maleic acid, oxalic acid, stearic acid, orotic acid, sebacic acid or mixtures thereof

16. The extended release tablet according to claim 8, wherein the release controlling agent is selected from the group consisting of water soluble/swellable polymers or mixtures thereof.

17. The extended release tablet according to claim 16, wherein the release controlling agent is selected from the group consisting of cellulose derivatives, gums, vinyl alcohol or vinylpyrrolidone-based polymers or mixtures thereof

18. The extended release tablet according to claim 17, wherein the release controlling agent is selected from the group consisting of hydroxypropyl methylcellulose, hydroxyethyl cellulose, hydroxypropyl cellulose, carboxymethyl cellulose, sodium carboxymethyl cellulose, xanthan gum, karaya gum, locust bean gum, alginic acid, sodium alginate, polyvinyl alcohol, polyvinylpyrrolidone or mixtures thereof

19. A process for preparing the extended release tablet comprising the steps of:
a) mixing micronized desvenlafaxine base, at least one pH modifier and one or more release controlling agents;
b) granulating the mixture of step (a) with or without a binder solution, to form granules;
c) mixing the granules with at least one pharmaceutically acceptable excipient and/or with at least one release controlling agent, to form a blend;
d) compressing the blend to form the tablet and
e) optionally coating the tablet.

20. A process for preparing the extended release monolithic tablet comprising the steps of:
a) mixing micronized desvenlafaxine base, at least one pH modifier and one or more release controlling agents;
b) granulating the mixture of step (a) with or without a binder solution, to form granules;
c) mixing the granules with at least one pharmaceutically acceptable excipient and at least one release controlling agent to form a blend;
d) compressing the blend to form the tablet and
e) optionally coating the tablet.
